(19) United States
(12) Patent Application Publication
(10) Pub. No.: US 2008/0057104 A1
(43) Pub. Date: Mar. 6, 2008

(54) MATRIX METALLOPROTEINASE INHIBITOR DELIVERING DEVICES

(76) Inventor: Joseph Walker, Shoreview, MN (US)

Correspondence Address: SCHWEGMAN, LUNDBERG & WOESSNER, P.A., P.O. BOX 2938, MINNEAPOLIS, MN, 55402, US

(21) Appl. No.: 11/469,592

(22) Filed: Sep. 1, 2006

(51) Int. Cl. A61F 2/02

(52) U.S. Cl. 424/426

(57) ABSTRACT

Devices, systems and methods which employ inhibitors of particular matrix metalloproteinases (MMPs) are provided.

### BACKGROUND

The heart is the center of a person's circulatory system. It includes an electro-mechanical system performing two major pumping functions. The heart includes four chambers: right atrium (RA), right ventricle (RV), left atrium (LA), and left ventricle (LV). The left portions of the heart, including LA and LV, draw oxygenated blood from the lungs and pump it to the organs of a body to provide the organs with their metabolic needs for oxygen. The right portions of the heart, including RA and RV, draw deoxygenated blood from the organs of the body and pump it to the lungs where the blood gets oxygenated. The efficiency of the pumping functions, indicative whether the heart is normal and healthy, is indicated by measures of hemodynamic performance, such as parameters related to intracardiac blood pressures and cardiac output.

In a normal heart, the sinoatrial node, the heart's natural pacemaker, generates electrical impulses, called action potentials, that propagate through an electrical conduction system to various regions of the heart to excite the myocardial tissues of these regions. Coordinated delays in the propagations of the action potentials in a normal electrical conduction system cause the various portions of the heart to contract in synchrony to result in efficient pumping functions indicated by a normal hemodynamic performance. A blocked or otherwise abnormal electrical conduction and/or deteriorated myocardial tissue cause dysynchronous contraction of the heart, resulting in poor hemodynamic performance, including a diminished blood supply to the organs of the body. The condition where the heart fails to pump enough blood to meet the body's metabolic needs is known as heart failure.

The adult myocardium is incapable of repairing itself after an injury. Such an injury may result from, for example, myocardial infarction (MI), which is the necrosis of portions of the myocardial tissue resulted from cardiac ischemia, a condition in which the myocardium is deprived of adequate oxygen and metabolite removal due to an interruption in blood supply. The adult heart lacks a substantial population of precursor, stem cells, or regenerative cells. Therefore, after the injury, the heart lacks the ability to effectively regenerate cardiomyocytes to replace the injured cells of the myocardium. Each injured area eventually becomes a fibrous scar that is non-conductive and non-contractile. Consequently, the overall contractility of the myocardium is weakened, resulting in decreased cardiac output. As a physiological compensatory mechanism that acts to increase the cardiac output, the LV diastolic filling pressure increases as the pulmonary and venous blood volume increases. This increases the LV preload, including the stress on the LV wall before the LV contracts to eject blood. The increase of the LV preload leads to progressive change of the LV shape and size, a process referred to as remodeling. Remodeling is initiated in response to a redistribution of cardiac stress and strain caused by the impairment of contractile function in the injured tissue as well as in nearby and/or interspersed viable myocardial tissue with lessened contractility due to the infarct. The remodeling starts with expansion of the region of the injured tissue and progresses to a chronic, global expansion in the size and change in the shape of the entire LV. Although the process is initiated by the compensatory mechanism that increases cardiac output, the remodeling ultimately leads to further deterioration and dysfunction of the myocardium. Consequently, the myocardial injury, such as resulted from MI, results in impaired hemodynamic performance and a significantly increased risk of developing heart failure.

What is needed is a method and device to inhibit ventricular remodeling post-MI.

### SUMMARY

The systemic side-effects of many compounds make their widespread use, e.g., to prevent, inhibit or treat post-infarct expansion and ventricular remodeling in a mammal post-MI, unwarranted. The present invention provides drug delivery devices, such as a stent, catheter, lead, or any combination thereof, in combination with one or more inhibitors of one more matrix metalloproteinases (MMPs) to allow for localized delivery to a desired area, such as an infarct region, without systemic side-effects. Such a drug/device combination may prevent or inhibit post-infarct expansion and subsequent ventricular remodeling. For example, one or more MMP inhibitors that are eluted from a drug eluting stent may allow for the beneficial effects of these compounds locally to the infarct region (e.g., directly upstream of an infarct) without the adverse effects of a systemic dose. Moreover, sustained release formulations having one or more MMP inhibitors can elute from the stent resulting in long term administration (chronic delivery) within the coronary circulation. In addition, one or more MMP inhibitors may be introduced via a catheter during an acute infarction period when positioned for deployment of a stent. Further, one or more MMP inhibitors may be delivered acutely and/or chronically through the lumen of an over-the-wire LV pacing lead. This localized delivery may be used to augment the anti-remodeling benefits of post-MI pacing with the LV lead positioned in the infarct region. Thus, both acute and chronic MMP inhibitor administration is envisioned.

The invention thus provides an implantable device configured to be positioned in or near the heart and to locally deliver one or more inhibitors of one or more MMPs to a treatment site. In one embodiment, the one or more inhibitors inhibit at least one of MMP-1, MMP-2, MMP-8, MMP-13, or MT-1. In one embodiment, the implantable device includes a stent having one or more MMP inhibitors applied to (coated on) and/or embedded in the matrix of the stent. In one embodiment, the implantable device include an epicardial patch having one or more MMP inhibitors applied to (coated on) and/or embedded in the matrix of the patch. In another embodiment, the implantable device includes an endocardial lead, e.g., useful to treat septal infarcts. In yet another embodiment, the implantable device includes a coronary venous (left side) lead.

Also provided is a method for treating a myocardial region including at least a portion of an injured area. The method includes delivering pacing pulses to the myocardial region of a mammal through one or more electrodes of a plurality of pacing electrodes on a lead, and delivering one or more inhibitors of one or more MMPs through a lumen in the lead in an amount effective to prevent or inhibit remodeling.

The invention also provides a method where pacing pulses are delivered to the myocardial region of a mammal through one or more electrodes of a plurality of pacing electrodes on a lead, and one or more inhibitors of one or more MMPs are delivered through a lumen in the lead in an amount effective to enhance pacing.

Further provided is a method in which a stent coated with one or more MMPs is delivered to a mammal via a catheter, and one or more inhibitors of one or more MMPs are delivered via a lumen in the catheter.

The invention also provides a system for a heart having a myocardial infarct region. The system includes an implantable agent delivery device adapted to release, and optionally contain, one or more inhibitors of one or more MMPs to a cardiac region including at least a portion of a myocardial infarct region; and an implantable cardiac rhythm management (CRM) device. The device includes a pacing circuit to deliver pacing pulses to the cardiac region, and a pacing controller adapted to control the delivery of the pacing pulses.

In one embodiment, the MMP inhibitor employed in the devices, systems and methods of the invention is an inhibitor of one or more of MMP-1, MMP-2, MMP-8, MMP-13, and/or MT-1. In one embodiment, the MMP inhibitor employed in the devices, systems and methods of the invention is a selective inhibitor of one or more of MMP-1, MMP-2, MMP-8, MMP-13, and/or MT-1, e.g., the inhibitor inhibits MMP-2 at least 2-fold, e.g., 10-fold or more, better than another MMP, e.g., MMP-7.

### BRIEF DESCRIPTION OF THE DRAWINGS

FIG. 1 is an illustration of an embodiment of an implantable lead.

FIG. 2 is an illustration of an embodiment of an implantable lead with a distal end including a coating carrying a MMP inhibitor.

FIG. 3 is an illustration of an embodiment of an implantable lead with a distal end including a matrix carrying a MMP inhibitor.

FIG. 3a is an illustration of an embodiment of an implantable lead with a distal end including an osmotic pump carrying a MMP inhibitor.

FIG. 4 is an illustration of an embodiment of an implantable lead with a distal end including a collar carrying a MMP inhibitor.

FIG. 4a is an illustration of an embodiment of an implantable lead with a distal end including a controlled pump having a reservoir carrying a MMP inhibitor.

FIG. 4b is an illustration of an embodiment of an implantable lead with a distal end including a matrix having a MMP inhibitor the delivery of which is controlled by electrophoresis.

FIG. 5 is an illustration of an embodiment of a stent carrying a MMP inhibitor mounted on an expandable member of a conventional catheter assembly.

FIG. 6 is an illustration of an embodiment of a stent and expandable member in an expanded state.

FIG. 7 is an illustration of an embodiment of a stent with an expandable member removed.

FIG. 8 is an illustration of an embodiment of a catheter including a lumen configured for injection of a MMP inhibitor.

FIG. 9 is an illustration of an embodiment of a catheter with a distal end including a coating carrying a MMP inhibitor.

FIG. 10 is an illustration of an embodiment of a catheter with a distal end including a drug collar carrying a MMP inhibitor.

FIG. 11 is an illustration of an embodiment of a catheter for angioplasty including a lumen configured for injection of a MMP inhibitor.

FIG. 12 is an illustration of an embodiment of a catheter for angioplasty with a distal end including a coating carrying a MMP inhibitor.

FIG. 13 is an illustration of an embodiment of a catheter for angioplasty with a distal end including a drug collar carrying a MMP inhibitor.

FIG. 14 is an illustration of an embodiment of a heart patch carrying a MMP inhibitor.

### DETAILED DESCRIPTION OF THE INVENTION

### LV Remodeling and ECM

LV myocardial remodeling that occurs in various settings of congestive heart failure (CHF) has historically been attributed to intrinsic changes in the cardiac myocyte. However, it is now recognized that changes also occur within the extracellular matrix (ECM) of the myocardium, contributing to the remodeling process. The myocardial ECM contains a fibrillar collagen network, a basement membrane, proteoglycans and glycosaminoglycans, and bioactive signaling molecules. The myocardial fibrillar collagens, such as collagen types I and III, ensure structural integrity of the adjoining myocytes, provide the means by which myocyte shortening is translated into overall LV pump function, and are essential for maintaining alignment of the myofibrils within the myocyte through a collagen-integrin-cytoskeletal myofibril relation (e.g., see Sackner-Bernstein, Curr. Cardiol. Rep., 2:112 (2000) and Burlew and Weber, Cardiol. Clin., 18:435 (2000)). The ECM forms a continuum between different cell types within the myocardium and provides a structural supporting network to maintain myocardial geometry during the cardiac cycle. Native ECM is continuously formed and then degraded by matrix metalloproteinases (MMPs) which along with their natural antagonists, the tissue-inhibiting metalloproteinases, regulate and determine the matrix turnover in living tissue.

### MMPs

MMPs play a pivotal role in normal tissue remodeling processes, such as tissue morphogenesis and wound healing (Woessner, In: Matrix Metalloproteinases, Parks (eds.), Academic Press, San Diego, Calif., pp. 1-14 (1998); Woessner and Nagase, In: Matrix Metalloproteinases and TIMPs, Oxford University Press, New York, N.Y., pp. 1-10 (2000); Vu and Werb, Genes Dev., 14:2123 (2000); Nelson et al., J. Clin. Oncol., 18:1135 (2000); Birkedal-Hansen et al., Crit. Rev. Oral. Biol. Med., 4:197 (1993); McDonnell et al., Biochem. Soc. Trans., 27:734 (1999)). This proteolytic system degrades a wide spectrum of ECM proteins and is constitutively expressed in a large number of cell and tissue types. Although MMPs likely play important roles in normal tissue remodeling, increased MMP expression has been identified in pathological processes, such as tumor angiogenesis and metastasis, rheumatoid arthritis, vascular neointimal hyperplasia, and plaque rupture (Nelson et al., J. Clin. Oncol., 18:1135 (2000); Birkedal-Hansen et al., Crit. Rev. Oral. Biol. Med., 4:197 (1993); McDonnell et al., Biochem. Soc. Trans., 27:734 (1999)). The MMPs constitute a family of zinc-dependent enzymes with over 20 members (Woessner, 1998; Woessner and Nagase, 2000).

There are two principal types of MMPs: those that are secreted into the extracellular space and those that are membrane bound (Table 1). The secreted MMPs are classified into several families based on their domain structure: matrilysin (minimal domain, MMP-7), collagenase (hemopexin domain, MMP-1, MMP-8, MMP-13), gelatinase (fibronectin domain, MMP-2, MMP-9), stromelysin (hemopexin domain, MMP-3, MMP-10, MMP-11), metalloelastase (MMP-12). The secreted MMPs constitute the majority of known MMPs and are released into the extracellular space in a latent or proenzyme state (proMMP). Activation of these latent MMPs is required for proteolytic activity, which can be achieved through enzymatic cleavage of the propeptide domain. Serine proteases, such as plasmin, as well as other MMP species can convert proMMPs to active enzymes (Woessner and Nagase, 2000; Murphy, Matrix Biol., 15:511 (1997)). Rapid amplification of MMP activity can thus occur after an initial enzymatic step. The cleavage of the propeptide domain results in a conformational change and exposure of the catalytic domain to the ECM substrate. There is a significant degree of homology within the catalytic domain of MMPs, and substrate specificity (see Table 2) is determined by the large extracellular binding domain at the C-terminus of the enzyme (Woessner, 1998; Woessner and Nagase, 2000; Knauper and Murphy, In: Matrix Metalloproteinases, Parks Ltd. (eds.), Academic Press, San Diego, Calif., pp. 199-218 (1998)).

TABLE 1
Classification of the MMP Family of Enzymes

| | Common Name |
|---|---|
| Collagenases | |
| MMP-1 | Collagenase-1 (Type 1, interstitial) |
| MMP-8 | Neutrophil collagenase |
| MMP-13 | Collagenase-3 |
| MMP-18 | Collagenase-4 |
| Gelatinases | |
| MMP-2 | Gelatinase-A (72 kDa) |
| MMP-9 | Gelatinase-B (92 kDa) |
| Stromelysins | |
| MMP-3 | Stromelysin-1, Proteoglycanase |
| MMP-10 | Stromelysin-2 |
| MMP-11 | Stromelysin-3 |
| MMP-7 | Matrilysin-1, PUMP |
| MMP-26 | Matrilysin-2, Endometase |
| MT-MMP | |
| (membrane type) | |
| MMP-14 | MT1-MMP |
| MMP-15 | MT2-MMP |
| MMP-16 | MT3-MMP |
| MMP-17 | MT4-MMP |
| MMP-24 | MT5-MMP |
| MMP-25 | MT6-MMP, Leucolysin |
| Other Enzymes | |
| MMP-12 | Macrophage metalloelastase |
| MMP-19 | RASI-1 |
| MMP-20 | Enamelysin |
| MMP-23 | CA-MMP |
| MMP-28 | Epilysin |

Activated MMPs undergo autocatalysis, resulting in lower molecular weight forms and, ultimately, in inactive protein fragments (Woessner and Nagase, 2000; Murphy et al., 1997). Another control point of MMP activity is through the presence of an endogenous class of low-molecular-weight molecules called TIMPs (Edwards et al., Int. J. Obes. Relat. Metab. Disord., 20:9 (1996); Woessner and Nagase, 2000; Baker et al., J. Clin. Invest., 101:1478 (1998); Greene et al., J. Biol. Chem., 271:30375 (1996); Li et al., Cardiovasc. Res., 42:162 (1999)). Different TIMP species have been identified and bind to activated MMPs in a 1:1 stoichiometric ratio. Certain TIMPs bind to proMMPs and thereby form MMP:TIMP complexes. TIMP-2 forms a complex with membrane-type MMPs and that this complex enhances the activation of proMMP (Murphy et al., 1997). In addition to binding to MMPs, TIMPs appear to influence cell growth and metabolism in vitro (Baker et al., J. Clin. Investig., 101:1478 (1998); Greene et al., 1996).

TABLE 2
The Names and Substrates of the MMP Family of Enzymes

| MMP | Other names | Substrates |
|---|---|---|
| MMP-1 | Collagenase-1; | Collagen type I, II, III, VII, VIII, X; |
| | interstitial collagenase | gelatin; aggrecan; MMP-2; MMP-9 |
| MMP-2 | Gelatinase A; 72 kDa | Collagen type I, II, III, IV, V, VII, |
| | gelatinase; type IV | X, XI, XIV; gelatin; aggrecan; |
| | collagenase; MMP-5 | laminin; fibronectin; elastin; |
| | | MMP-9; MMP-13 |
| MMP-3 | Stromelysin-1; pro- | Collagen type II, III, IV, |
| | collagenase activator; | Invention(s), X, XI; gelatin; |
| | transin-1; MMP-6 | aggrecan, laminin; fibronectin; |
| | | elastin; MMP-1, -7, -8, -9, and -13 |
| MMP-7 | Matrilysin-1; matrin; | Collagen type IV, X; gelatin; |
| | PUMP-1; uterine | aggrecan, laminin; fibronectin; |
| | metalloendopeptidase | elastin; MMP-1, -2, and -9 |
| MMP-8 | Collagenase-2; | Collagen type I, II, III, V, VII, VIII, |
| | neutrophil collagenase | X; gelatin; aggrecan; laminin; |
| | | fibronectin; elastin |
| MMP-9 | Gelatinase B; 92 kDa | Collagen type IV, V, VII, X, XIV; |
| | gelatinase; type IV | gelatin; aggrecan; fibronectin; |
| | collagenase | elastin |
| MMP-10 | Stromelysin-2; | Collagen type III, IV, V; gelatin; |
| | transin-2 | aggrecan; laminin; fibronectin; |
| | | elastin; MMP-1; MMP-8 |
| MMP-11 | Stromelysin-3 | Aggrecan; fibronectin; laminin; |
| | | α-1 antitrypsin |
| MMP-12 | Macrophage | Collagen IV; elastin; gelatin; |
| | metalloelastase | laminin; fibronectin; vitronectin |
| MMP-13 | Collagenase-3 | Collagen type I, II, III, IV; gelatin; |
| | | aggrecan; MMP-9 |
| MMP-14 | MT1-MMP | Collagen type I, II, III; gelatin; |
| | (membrane-type-1 | aggrecan; laminin; fibronectin; |
| | MMP) | elastin; MMP-2; MMP-13 |
| MMP-15 | MT2-MMP | Gelatin; laminin; fibronectin; |
| | | MMP-2 |
| MMP-16 | MT3-MMP; ovary | MMP-2 |
| | metalloproteinase | |
| MMP-17 | MT4-MMP; | Fibrin; fibrinogen; TNF precursor |
| | stromelysin A; | |
| | stromelysin B | |
| MMP-18 | Xenopus collagenase-4 | Unknown |
| MMP-19 | RASI-1; RASI-6 | Gelatin; aggrecan; COMP; |
| | | collagen type IV; laminin; nidogen; |
| | | large tenas |
| MMP-20 | Enamelysin | Amelogenin; aggrecan; COMP |
| MMP-21 | Xenopus MMP; | Unknown |
| | XMMP | |
| MMP-22 | Gallus domesticus | Gelatin; casein |
| (MMP-27) | MMP; CMMP | |
| MMP-23 | CA-MMP | McaPLGLDpaARNh2 (synthetic |
| | | MMP substrate) |
| MMP-24 | MT5-MMP | MMP-2 |
| MMP-25 | MT6-MMP; leukolysin | Gelatin |
| MMP-26 | Matrilysin-2; | Collagen type IV; gelatin; α₁-PI; |
| | endometase | fibronectin; fibrinogen; pro-MMP-9 |
| MMP-28 | Epilysin | Casein |

The transmembrane domain family (MT-MMPs) includes MMP-14 through MMP-17. Because MT-MMPs are membrane bound, they provide a focalized area for ECM proteolytic degradation. During trafficking to the cell membrane, MT-MMPs undergo intracellular activation through a proprotein convertase pathway (Murphy et al., 1997; Miyamori et al., Biochem. Biophys. Res. Commun., 267:796 (2000)). Thus, unlike other classes of MMPs, MT-MMPs are proteolytically active once inserted into the cell membrane. MT-MMPs contain a substrate recognition site for other MMP species and so constitute an important pathway for activation of other MMPs within the ECM (Woessner and Nagase, 2000; Murphy et al., 1997). MT1-MMP degrades fibrillar collagens and a wide range of ECM glycoproteins and proteoglycans. Moreover, MT1-MMP proteolytically processes the proforms of the gelatinase MMP-2 and the interstitial collagenase MMP-13. The MT-MMPs do not appear to be under the influence of local inhibitory control because the tissue inhibitors of the MMPs (TIMPs) apparently fail to effectively bind to MT-MMPs (Miyamori et al., 2000). MT-MMPs appear to be expressed in both normal and diseased cells (Miyamori et al., 2000; Shimada et al., Eur. J. Biochem., 262:907 (1999); Kajita et al., FEBS Lett., 457:353 (1999); Llano et al., Cancer Res., 59:2570 (1999); Velasco et al., Cancer Res., 60:877 (2000); Goldberg et al., Proc. Natl. Acad. Sci. USA, 86:8207 (1989)). A number of cell types within the myocardium express MT-MMPs, which include fibroblasts, vascular smooth muscle, and cardiac myocytes.

### LV Dysfunction and MMPs

LV regional myocardial dysfunction and remodeling that occur immediately after MI can persist long after the acute insult (Pfeffer et al., Circulation, 81:1161 (1990); Chareonthaitawee et al., J. Am. Coll. Cardiol., 25:567 (1995); St. John Sutton et al., Circulation, 96:3294 (1997); Jugdutt et al., Clin. Cardiol., 10:641 (1987); St. John Sutton et al., Circulation, 101:2981 (2000); Jugdutt, J. Am. Coll. Cardiol., 25:1718 (1995)). The summation of cellular and extracellular events that occur in the post-MI period results in changes in LV geometry and has been called “infarct expansion.” Past studies have demonstrated that a structural determinant of infarct expansion is extracellular remodeling (Pfeffer et al., 1990; St. John Sutton et al., 2000; Jugdutt, 1995). MMPs have been implicated in tissue remodeling (Sun et al., Cardiovasc. Res., 46:250 (2000). For instance, increased MMP expression has been reported in patients with end-stage heart failure and in several animal models of developing LV dysfunction (Thomas et al., Circulation, 97:1708 (1998); Thomas et al., Circulation, 97:1708 (1998); Li et al., Circulation, 98:1728 (1998); Spinale et al., Circulation, 102:1944 (2000); Coker et al., Am. J. Physiol., 277:777 (1999); Peterson et al., Cardiovasc. Res., 46:307 (2000); Spinale et al., Circ. Res., 85:364 (1999); Li et al., Cardiovasc. Res., 46:298 (1999); Peterson et al., Circulation, 103:2303 (2001); Rohde et al., Circulation, 99:3063 (1999)). Increased interstitial MMP activity has been demonstrated to occur directly within the ischemic myocardium (Etoh et al., Am. J. Physiol., 281:987 (2001)).

Experimental studies using pharmacological compounds that inhibit all MMPs (broad-spectrum inhibitors) have been demonstrated to directly affect LV remodeling after MI (Rohde et al., 1999; Mukherhee et al., Circulation, 107:618 (2003); Creemers et al., Circ. Res., 89:201 (2001)). However, whether broad-spectrum MMP inhibition is necessary to favorably modulate LV remodeling after MI remains unclear. Moreover, early MMP inhibition may adversely affect normal wound-healing responses (Creemers et al., 2001; Heymans et al., 1999; Ducharme et al., 2000).

### Methods and Devices of the Invention

The invention provides for methods, devices and systems having the devices, useful to prevent, inhibit or treat post-infarct expansion and/or ventricular remodeling, or enhance the efficacy of post-infarct pacing, or any combination thereof. The methods, devices, and systems employ one or more inhibitors of one or more MMPs. In one embodiment, one or more broad spectrum inhibitors of MMPs are employed. In another embodiment, one or more selective inhibitors of one more MMPs are employed. In one embodiment, one or more broad spectrum inhibitors of MMPs are delivered during device delivery (acute delivery). In another embodiment, one or more selective inhibitors of one or more MMPs are delivered during device delivery (acute delivery). In yet another embodiment, one or more broad spectrum inhibitors of MMPs are delivered chronically. In a further embodiment, one or more selective inhibitors of one or more MMPs are delivered chronically. For example, a stent may be employed that delivers a MMP inhibitor upon stent placement (acute delivery) and which optionally may contain a MMP inhibitor, either the same or a different MMP inhibitor, for sustained release which is present in a sustained release formulation coated on the stent (chronic delivery). Alternatively, a stent may contain a MMP inhibitor for sustained release which is present in a sustained release formulation. In this embodiment, a catheter or lead may be employed to deliver a MMP inhibitor during or soon after an infarct (acute delivery). In another embodiment, a catheter or lead may be employed to deliver a MMP inhibitor during or soon after an infarct in the absence of stent placement or in conjunction with stent placement, which stent may contain a drug that is not a MMP inhibitor or may be a stent that does not itself deliver a drug.

### Exemplary MMP Inhibitors

There are three major components to most endogenous MMP inhibitors (TIMPs): the zinc binding group ZBG, the peptidic backbone and the pocket occupying side chain. Most MMPs inhibitors are classified according to their ZBG. Inhibitors interactions at active-site zinc play a role in defining the binding mode and relative inhibitor potency. MMP inhibitors generally contain an effective zinc binding group (e.g., hydroxamic acid, carboxylic acid, or sulfhydryl group) that is either generally substituted with a peptide-like structure that mimics the substrates that they cleave or appended to smaller side chains that may interact with specific subsites (e.g., P1′, P2′, P3′) within the active site.

Exemplary MMP inhibitors useful in the devices, methods and systems of the invention include but are not limited to TIMPs and compounds disclosed in U.S. Pat. Nos. 6,890,937, 6,750,233, 6,541,489, 6,872,727, 6,794,511, 6,750,228, 6,747,027, 6,716,844, 6,656,954, 6,638,952, 6,624,144, 6,583,299, 6,492,367, 6,476,027, 6,451,791, 6,448,250, 6,130,254, 6,087,559, 6,013,649, 5,990,158, 5,804,593, 5,948,780, 5,270,958, 5,240,958, 4,595,700, 6,420,408, 6,350,885, 6,265,432, and 6,116,910, in U.S. published applications 20040138260, 20060074108, and 20010049449 and in Wada, Curr. Topics in Med. Chem., 4:1255 (2004) and Skiles et al., Curr. Med. Chem., 11:2911 (2004)), the disclosures of which are incorporated by reference herein. Other exemplary MMP inhibitors include but are not limited to batimistat (BB-94), marimistat (BB-2516), prinomastat (AG3340), N-formylhydroxylamine (retrohydroxamate) biaryl ethers, e.g., ABT-770, BAY129566, minocylcine, doxocycline, tetracycline, doxycycline, methacycline, oxytetracycline, demeclocycline, 6-demethyl-6-deoxy-4-de(dimethylamino)tetracycline, 6-deoxy-5-hydroxy-4-de(dimethylamino)tetracycline, retinoids, antioxidants, e.g., glutathione, N-acetyl cysteine, glutathione ethyl ester, BMS-275291, R032-3555 (Trocade), bryostatin, HMG CoA reductase inhibitors, e.g., statins, ONO-4817 (Shimoyama et al., Med. Sci. Monit., 12:BR51 (2006)), allylisothiocyanate, GM 6001 (Dwiovedi et al., Am. J. Path., 168: 69 (2006), gallocatechin-3-gallate, tricyclic sulfonamides, heteroaromatics, 2-substituted oxazoles (Sheppart et al., Bioorg. Med. Chem. Lett., 8:3251 (1998), and thiol group-containing amide or peptidyl amide based MMPIs disclosed in WO 95/12389 and WO 96/11209. In one embodiment, a selective MMP inhibitor is employed. In one embodiment, the selective MMP inhibitor inhibits MMP-2, e.g., the inhibitor is/has a hydroxamic acid. In another embodiment, the selective MMP inhibitor inhibits MMP-8, e.g., the inhibitor is/has a carboxylic acid, thiadazin, barbiturate ring, phosphonic acid, peptidic thiol or hydroxamic acid such as a peptidic hydroxamic acid or malenic acid based hydroxamic acid, including BB94. In another embodiment, the selective MMP inhibitor inhibits MMP-13, e.g., the inhibitor is/has a hydroxamic acid, such as CGS 270237A, or a sulfone hydroxamic acid, or hemopexin. In one embodiment, the MMP inhibitor is one in one of Tables 5-12 in Skiles et al., Curr. Med. Chem., 11:2911 (2004), the disclosure of which is incorporated by reference herein.

### Devices

Referring now to the drawings, there are shown in the Figures various implantable devices configured to carry and deliver one more inhibitors of one or more MMPs to a local area within a body. Those areas in the body that would benefit from the one or more inhibitors of the one or more MMPs include those areas that experience remodeling after infarct and those areas that experience trauma during surgical implantation of a device.

The implantable devices include, but are not limited to, cardiac rhythm management (CRM) device leads, stents, catheters, mechanical heart valves, atrial septal defect (ASD) devices, heart patches and ventricular restraint devices (VRD). Some of these devices, such as leads, heart patches, stents and catheters, may be implanted at or near an infarct resulting from an ischemia event. Devices such as stents, catheters, heart patches and leads may be implanted in or near the heart in reaction to an ischematic event or other cardiac event or disorder. Devices such as stents, heart patches or leads may be used for acute or chronic delivery, or both, and catheters may be used for acute delivery of MMP inhibitors.

Various methods of medicating the implantable devices are described below. While each of these methods is described in association with particular embodiments of the invention, it is understood that the various methods of medicating may be used with other embodiments, depending on the composition of the device. For example, methods of medicating devices having components made of silicone rubber are described in association with CRM device leads. Such methods may, however, be equally applicable to VRDs and cardiac patches. Also, methods of medicating metallic and polymeric components are also described in association with CRM device leads. These methods may find application in other devices such as stents, valves and heart patches.

### Exemplary Coatings and Drug Delivery Vehicles

Device coating may be accomplished by dipping the device in a solution or by spraying the device, or other methods of applying a coating to a device.

Non-thrombogenic and anti-thrombogenic coatings for devices have been developed, e.g., devices coated with polymers having pendant zwitterionic groups, specifically phosphorylcholine (PC) groups, generally described in WO 93/01221, or those described in WO 98/30615. The polymers coated onto the device have pendant crosslinkable groups which are subsequently crosslinked by exposure to suitable conditions, generally heat and/or moisture. Specifically a trialkoxysilylalkyl group reacts with pendant groups of the same type and/or with hydroxyalkyl groups to generate intermolecular crosslinks, which may lead to reduced thrombogenicity. Other drug coatings are described in Topol and Serruys in Circulation, 98:1802, (1998) and McNair et al., Proceedings of the International Symposium on Controlled Release Bioactive Materials, pp. 338-339 (1995) (hydrogel polymers having pendant phosphorylcholine groups). The hydrophilic/hydrophobic ratio of the (hydrophilic) phosphorylcholine monomer 2-methacryloyloxyethyl phosphorylcholine (HEMA-PC) and a hydrophobic comonomer may be modified. Crosslinking may be achieved by incorporating a reactive monomer 3-chloro-2-hydroxypropylmethacrylate. Release rates of drugs are influenced by the molecular size, solute partitioning and degree of swelling of the polymer.

Other coatings include polyurethanes. The polyurethanes may be modified to control compatibility with lipophilic or hydrophilic drugs. A polyurethane coated device may be contacted with a drug in a solvent which swells the polyurethane, whereby drug is absorbed into the polyurethane. Selection of a suitable solvent takes into account the swellability of the polyurethane and the solubility of the drug in the solvent.

Coatings for implantable devices may include an undercoat having a particulate drug and polymer matrix, and an overlying topcoat which partially covers the undercoat. The top coat may be discontinuous in situ, in order to allow for release of the drug from the undercoat. The polymer of the undercoat is, for example, hydrophobic biostable elastomeric material such as silicones, polyurethanes, ethylene vinyl acetate copolymers, polyolefin elastomers, polyamide elastomers and EPDM rubbers. The top layer may be formed of non-porous polymer such is as fluorosilicones, polyethylene glycols, polysaccharides and phospholipids.

Polymers having metal chelating activities may also have MMP inhibitory activity, e.g., polymers capable of chelating divalent metals. Those polymers are generally polymers of unsaturated carboxylic acids although sulphonated anionic hydrogels may be used. One example of a monomer for forming a sulphonated anionic hydrogel is N,N-dimethyl-N-methacryloyloxy-ethyl-N-(3-sulphopropyl) ammonium betaine. Other examples of polymers are acrylic acid based polymers modified with C₁₀₋₃₀-alkyl acrylates crosslinked with di- or higher-functional ethylenically unsaturated crosslinking agents. In one embodiment, a device is coated with a crosslinkable polymer of 2-methacryloyloxyethyl-2′-trimethyl ammoniumethylphosphate inner salt and dodecyl methacrylate with crosslinking monomer.

Curing of a crosslinkable polymer may involve exposure to irradiation, chemical curing agents, catalysts or, more usually raised temperature and/or reduced pressure to acceptable condensation based cross-linking reactions. Drying a liquid composition usually involves raised temperature and/or reduced pressure for a time sufficient to reduce the amount of solvent remaining on the device to undetectable levels or levels at which it will not interfere with subsequent processing steps, or with release of the drug in use, or be toxic to a patient in whom the device is implanted.

In one embodiment, the coating on the outer wall of the device includes an inner layer of an amphiphilic polymer and adhered to the inner layer a crystalline MMP inhibitor. Provision of the crystalline MMP inhibitor may also confer useful release characteristics on the device. The crystalline material may be controlled for a particle size, for instance, to confer desired release characteristics which complement the release of absorbed drug from a polymer coating.

In one embodiment, the coating on at least the outer wall of the device has an inner layer where the polymer is amphiphilic and the topcoat has a non-biodegradable, biocompatible semipermeable polymer. The semipermeable polymer is selected so as to allow permeation of the MMP inhibitor through the top layer when the device is in an aqueous environment. In such an environment, the semipermeable polymer may, for instance, be swollen, and it is in this form that it should allow permeation of the active MMP inhibitors. A topcoat may confer desirable controlled release characteristics. Its use is of particular value where coating comprises crystalline MMP inhibitor adhered to an inner layer of amphiphilic polymer. The topcoat in such an embodiment has several functions. It provides a smooth outer profile, minimizes loss of the MMP inhibitor during delivery, provides a biocompatible interface with the blood vessel after implantation and controls release of MMP inhibitor from the stent into the surrounding tissue in use. A topcoat is preferably substantially free of the MMP inhibitor prior to implantation of the device. A topcoat may be formed of a second cross-linked amphiphilic polymer. The second amphiphilic polymer may be the same as the first amphiphilic polymer.

A composition to be applied to an implantable component is prepared by conventional methods wherein all composition components are combined and blended. For example, a predetermined amount of a polymer is added to a predetermined amount of a solvent. The term polymer is intended to include a product of a polymerization reaction inclusive of homopolymers, copolymers, terpolymers, etc., whether natural or synthetic, including random, alternating, block, graft, crosslinked, hydrogels, blends, compositions of blends and variations thereof. The solvent can be any single solvent or a combination of solvents capable of dissolving the polymer. The particular solvent or combination of solvents selected is dependent on factors such as the material from which implantable device is made and the particular polymer selected. Representative examples of suitable solvents include, but are not limited to, aliphatic hydrocarbons, aromatic hydrocarbons, alcohols, ketones, dimethyl sulfoxide (DMSO), tetrahydrofuran (THF), dihydrofuran (DHF), dimethylacetamide (DMAC), acetates and combinations thereof.

Sufficient amounts of a MMP inhibitor or a combination thereof are then dispersed in the blended composition of the polymer and the solvent. The MMP inhibitor may be in true solution or saturated in the composition. If the MMP inhibitor is not completely soluble in the composition, operations such as gentle heating, mixing, stirring, and/or agitation can be employed to effect homogeneity of the residues. However, care should be taken to ensure that the use of heat to effect dissolution does not also cause denaturation of a heat-sensitive anti-apoptotic drug substance.

Alternatively, the MMP inhibitor substance may be encapsulated in a sustained delivery vehicle such as, but not limited to, a liposome or an absorbable polymeric particle. The preparation and use of such sustained delivery vehicles are well known to those of ordinary skill in the art. The sustained delivery vehicle containing the MMP inhibitor is then suspended in the composition.

Inclusion of the MMP inhibitor in the composition should not adversely alter the composition or characteristic of the MMP inhibitor. Accordingly, the particular MMP inhibitor is selected for mutual compatibility with the other components of the composition.

Details of methods of coating or impregnating metallic and/or polymeric components with drugs are described in the following patents: U.S. Pat. No. 6,287,628, titled “Porous Prosthesis and a Method of Depositing Substances into the Pores;” U.S. Pat. No. 6,506,437, titled “Methods of Coating an Implantable Device Having Depots Formed in a Surface Thereof;” U.S. Pat. No. 6,544,582, titled “Method and Apparatus for Coating an Implantable Device;” U.S. Pat. No. 6,555,157, titled “Method for Coating an Implantable Device and System for Performing the Method;” U.S. Pat. No. 6,585,765, titled “Implantable Device Having Substances Impregnated Therein and a Method of Impregnating the Same” and U.S. Pat. No. 6,616,765, titled “Apparatus and Method for Depositing a Coating onto a Surface of a Prosthesis.”

In one embodiment, the device is a stent made of a nonbiodegradable, biocompatible material such as shape memory metal, or may be elastically self-expanding, for instance, be a braided stent or a balloon expandable stent. In one embodiment of the invention, in which a topcoat is provided, the topcoat may be part of a coherent coating formed over both a stent and a stent delivery device, for instance, a balloon of a balloon catheter from which a balloon expandable stent is delivered. In this case, the balloon may additionally be provided with a coating having the MMP inhibitor, for instance, adsorbed onto parts of its exterior surface between stent struts. Such a device may be produced by loading the stent with the MMP inhibitor after the stent has been mounted onto the delivery catheter.

In one embodiment, contact of the polymer coated stent with a liquid MMP inhibitor composition may be by dipping the stent into a body of the stent, and/or by flowing, spraying or dripping a liquid composition onto the stent with immediate evaporation of solvent from the wet stent. Such steps allow good control of drug loading onto the stent, and are particularly useful for forming the crystals of drug at the surface of polymer.

While the stent may be provided with drug coating prior to being mounted onto its delivery device, the stent to be premounted onto its delivery device prior to coating the stent. In this embodiment, it is primarily the outer wall of the stent (as opposed to the inner wall of the stent) which becomes coated with the MMP inhibitor. This method generally results in the MMP inhibitor being coated onto the stent delivery section of the delivery catheter. The outer surface of the delivery catheter with a coating of a MMP inhibitor, is a source to deliver the inhibitor adjacent tissue upon placement of the stent. Generally the delivery catheter is in contact with such tissue for a short period, whereby contact is not maintained for a prolonged period, and limited level of transfer of drug from the balloon takes place.

In another embodiment of the invention, local delivery of the MMP inhibitor is achieved using a microparticle, polymeric matrix delivery system which releases the drug into surrounding tissue. Both non-biodegradable and biodegradable matrices can be used for delivery of the drug, although biodegradable matrices are preferred. These may be natural or synthetic polymers. The polymer is selected based on the period over which drug release is desired. The microparticles can be microspheres, where the drug is dispersed within a solid polymeric matrix, or microcapsules, where the core is of a different material than the polymeric shell, and the drug is dispersed or suspended in the core, which may be liquid or solid in nature.

Bioerodible microspheres can be prepared using any of the methods developed for making microspheres for drug delivery, for example, as described by Mathiowitz and Langer, J. Controlled Release, 5:13 (1987); Mathiowitz et al., Reactive Polymers, 6:275 (1987); and Mathiowitz et al., J. Appl. Polymer Sci., 35:755 (1988), the teachings of which are hereby incorporated by reference. The selection of the method depends on the polymer selection, the size, external morphology, and crystallinity that is desired, as described, for example, by Mathiowitz et al., Scanning Microscopy, 4:329 (1990); Mathiowitz et al., J. Appl. Polymer Sci., 45:125 (1992); and Benita et al., J. Pharm. Sci., 73:1721 (1984), the teachings of which are incorporated herein.

Delivery of the microspheres is facilitated by a catheter or lead placed in or near the treatment site. The tip of the catheter or lead is placed upstream from the target treatment site such that when the microspheres are released through the catheter tip, they disperse and lodge themselves in the treatment area.

Any one or more catheters may be used to deliver the one or more MMP inhibitors to the infarct region area. Several catheters have been designed in order to precisely deliver agents to a damaged region within the heart for example an infarct region. Several of these catheters have been described (U.S. Pat. Nos. 6,102,926, 6,120,520, 6,251,104, 6,309,370; 6,432,119; 6,485,481). The delivery device may include an apparatus for intracardiac drug administration, including a sensor for positioning within the heart, a delivery device to administer the desired agent and amount at the site of the position sensor. The apparatus may include, for example, a catheter body capable of traversing a blood vessel and a dilatable balloon assembly coupled to the catheter body comprising a balloon having a proximal wall. A needle may be disposed within the catheter body and includes a lumen having dimensions suitable for a needle to be advanced there through. The needle body includes an end coupled to the proximal wall of the balloon. The apparatus also includes an imaging body disposed within the catheter body and including a lumen having a dimension suitable for a portion of an imaging device to be advanced there through. The apparatus may further include a portion of an imaging device disposed within the imaging body adapted to generate imaging signal of the infarct region within the ventricle. The apparatus may be suitable for accurately introducing a treatment agent at a desired treatment site.

In another embodiment a needle catheter used to deliver the agent to the ventricle for example, the infarct region, may be configured to include a feedback sensor for mapping the penetration depth and location of the needle insertion. The use of a feedback sensor provides the advantage of accurately targeting the injection location. Depending on the type of agent administered, the target location for delivering the agent may vary. For example, one agent may require multiple small injections within an infarct region where no two injections penetrate the same site.

In other embodiments, the catheter assembly may include a maneuverable instrument. This catheter assembly includes a flexible assembly. The catheter assembly, may be deflectable and includes a first catheter, a second catheter, and a third catheter. The second catheter fits coaxially within the first catheter. At least one of the first catheter and the second catheter include a deflectable portion to allow deflection of that catheter from a first position to a second position, and the other of the first catheter and second catheter includes a portion which is preshaped (e.g., an angled portion formed by two segments of the angled portion). The third catheter has a sheath and a medical instrument positioned within the sheath. The third catheter fits coaxially within the second catheter. In another embodiment, a stabilizer, such as a donut shaped balloon, is coupled to a distal portion of the third catheter. Each catheter is free to move longitudinally and radially relative to the other catheters. The catheter assembly uses coaxially telescoping catheters at least one or more being deflectable, to position a medical instrument at different target locations within a body organ such as the left ventricle. The catheter assembly may be flexible enough to bend according to the contours of the body organ. The catheter assembly may be flexible in that the catheter assembly may achieve a set angle according to what the medical procedure requires. The catheter assembly will not only allow some flexibility in angle changes, the catheter assembly moves in a three coordinate system allowing an operator greater control over the catheter assembly's movement portion of the second catheter, allowing for the distal tip of the third catheter to be selectively and controllably placed at a multitude of positions. It will be appreciated that the deflectable portion may alternatively be on the second catheter and the preshaped portion may be on the first catheter.

In a further embodiment, an apparatus is disclosed. In one embodiment, the apparatus includes a first annular member having a first lumen disposed about a length of the first annular member, and a second annular member coupled to the first annular member having a second lumen disposed about a length of the second annular member, wherein collectively the first annular member and the second annular member have a diameter suitable for placement at a treatment site within a mammalian body. Representatively, distal ends of the first annular member and the second annular member are positioned with respect to one another to allow a combining of treatment agents introduced through each of the first annular member and the second annular member to allow a combining of treatment agents at the treatment site. Such an apparatus is particularly suitable for delivering a multi-component gel material (e.g., individual components through respective annular members that forms a bioerodable gel within an infarct region of a ventricle).

In the embodiments described herein, a substance delivery device and a method for delivering a substance are disclosed. The delivery device and method described are particularly suitable, but not limited to, local drug delivery in which a treatment agent composition (possibly including multiple-treatment agents and/or a sustained-release composition) is introduced via needle delivery to a treatment site within a mammalian host. A kit of a treatment agent composition is also described. One suitable application for a delivery device is that of a catheter device, including a needle delivery system. Suitable therapies include, but are not limited to, delivery of drugs for the treatment of arterial restenosis, therapeutic angiogenesis, or cancer treatment drugs/agents.

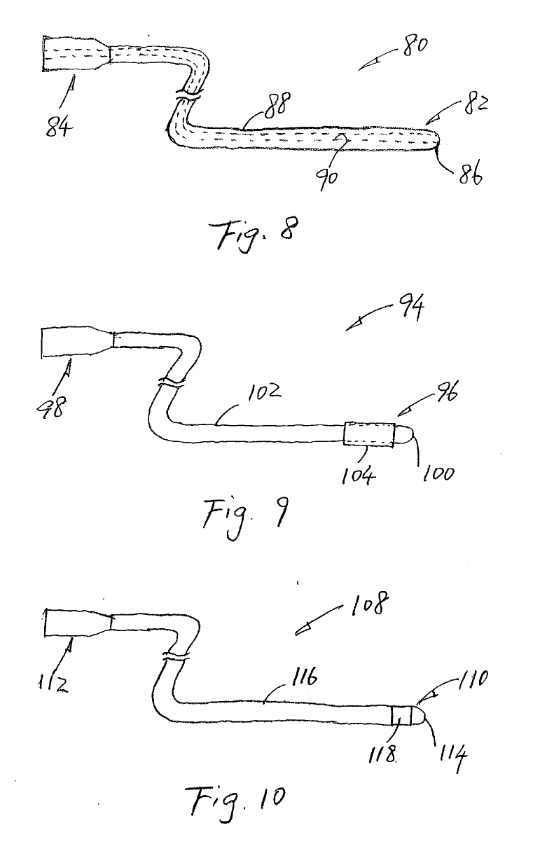

In various embodiments, an MMP inhibitor is locally delivered using an implantable or percutaneous device. Examples of such devices include leads, stents, and catheters, as discussed below with reference to FIGS. 8-13. In the discussion of these devices, “the MMP inhibitor” represents any of the MMP inhibitors or any combination of the MMP inhibitors that are discussed in this document.

### Leads

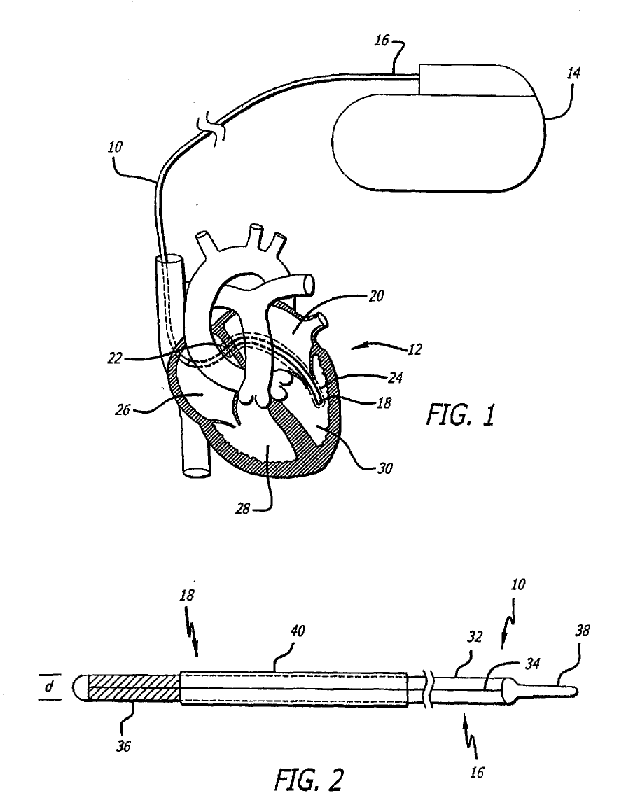

With reference to FIG. 1, in one embodiment, an implantable lead 10 provides for access to a chamber of a heart 12. The lead 10 is part of an implantable CRM device 14 and includes a proximal end 16, which is coupled to the device 14, and a distal end portion 18, which is coupled on or about one or more portions of the heart 12. In the illustrated embodiment, the lead 10 is a coronary sinus lead. The CRM device 14 may be implanted in response to a myocardial infarction and the lead 10 may be positioned to provide access to a myocardial region in or near an infarct. In other embodiments, the lead 10 is an epicardial lead providing access to an epicardial region of the heart 12, or an endocardial lead providing access to an endocardial region of the heart 12.

As illustrated in FIG. 1, the distal end portion 18 of the lead 10 is transvenously guided to a left ventricle 30, through a coronary sinus 22 and into a great cardiac vein 24. This positioning of the lead 10 is useful for delivering pacing and/or defibrillation energy to the left side of the heart 12 such as for treatment of cardiac disorders requiring therapy delivered to the left side of the heart 12. Other possible positions of the distal portion 18 of the lead 10 include insertion in to a right atrium 26 and/or a right ventricle, or transeptal insertion into a left atrium 20 and/or the left ventricle 30.

With reference to FIGS. 2-4, the distal end portion 18 of the lead 10 is configured to carry and locally deliver the MMP inhibitor to the area around the region in which the distal end portion 18 is placed. Thus, only those portions of the implanted lead in proximity to the heart 12 deliver the MMP inhibitor.

With reference to FIG. 2, in one embodiment, the lead 10 includes a biocompatible flexible insulating elongate body 32 (e.g., including a polymer such as medical grade silicone rubber) for transluminal (i.e., transvenous or transarterial) insertion and access within a living organism. In one embodiment, the slender elongate body 32 is tubular and has a peripheral outer surface of diameter d that is small enough for transluminal insertion into the coronary sinus 22 and/or great cardiac vein 24. An elongate electrical conductor 34 is carried within the insulating elongate body 32. The conductor 34 extends substantially along the entire length between the distal end portion 18 and proximal end 16 of the lead 10, and this length is long enough for the lead 10 to couple the device 14, which is implanted pectorally, abdominally, or elsewhere, to desired locations within the heart 12 for sensing intrinsic electrical heart activity signals or providing pacing/defibrillation-type therapy.

The elongate body 32 forms an insulating sheath covering around the conductor 34. The conductor 34 is coupled to a ring or ring-like electrode 36 at or near the distal end portion 18 of the elongate body 32. The conductor 34 is coupled to a connector 38 at or near the proximal end 16 of the elongate body 32. The device 14 includes a receptacle for receiving the connector 38, thereby obtaining electrical continuity between the electrode 36 and the device 14.

The electrode 36, or at least a portion thereof, is not covered by the insulating sheath of the elongate body 32. The electrode 36 provides an exposed electrically conductive surface around all, or at least part of, the circumference of the lead 10. In one example, the electrode 36 is a coiled wire electrode that is wound around the circumferential outer surface of the lead 10. The lead 10 also includes other configurations, shapes, and structures of the electrode 36.

As illustrated in FIG. 2, the lead 10 includes a biocompatible coating 40 on at least one insulating portion of the peripheral surface of the elongate body 36 at or near the distal end portion 18. The coating 40 extends circumferentially completely (or at least partially) around the tubular outer peripheral surface of the lead 10 and carries the MMP inhibitor. In use, when this lead 10 is inserted and implanted in the body, the coating 40 dissolves and the MMP inhibitor is released. The time duration of the release of the MMP inhibitor is preferably between several weeks and months. The time it takes for the coating 40 to fully dissolve and thus for the MMP inhibitor to be completely released may be controlled based on the selection of the coating material and the concentration of the MMP inhibitor.

In one embodiment, the coating 40 includes substantially soluble particles dispersed in a substantially insoluble medium, such as biocompatible silicone rubber medical adhesive, other polymer, or other suitable biocompatible adhesive substance. The soluble particles are at least partially dissolvable when exposed to an aqueous substance such as blood or bodily fluids. In accordance with the present invention, the soluble particles include the MMP inhibitor. The particles may also include a drug enhancer. When the coating 40 is exposed to an aqueous environment, the substantially soluble MMP inhibitor particles dissolve, providing sustained release of the MMP inhibitor into the surrounding tissue. During manufacture of the lead, one or more portions of the lead are coated with the coating. The coating cures such that it adheres to the lead. Details relating to the coating formation are described in U.S. Pat. No. 6,584,363, titled “Implantable Lead With Dissolvable Coating for Improved Fixation and Extraction,” assigned to Cardiac Pacemakers, Inc., the disclosure of which is hereby incorporated by reference.

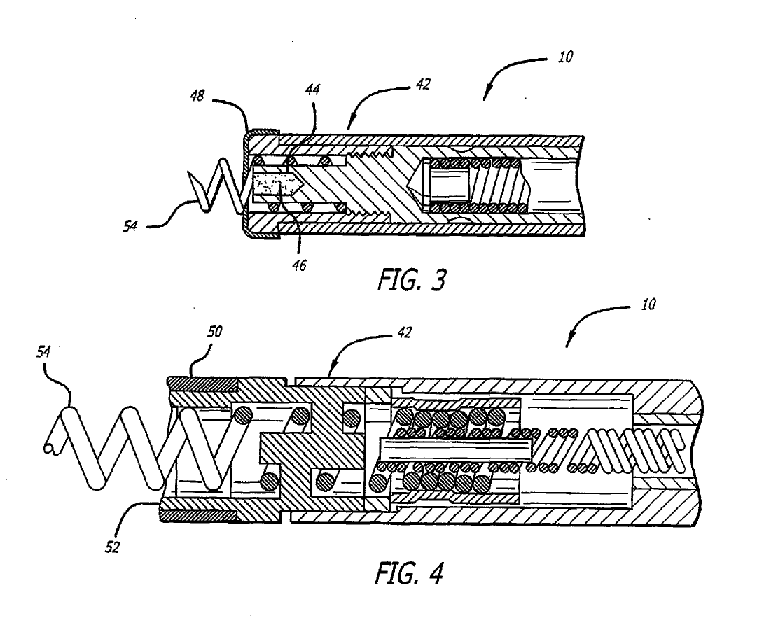

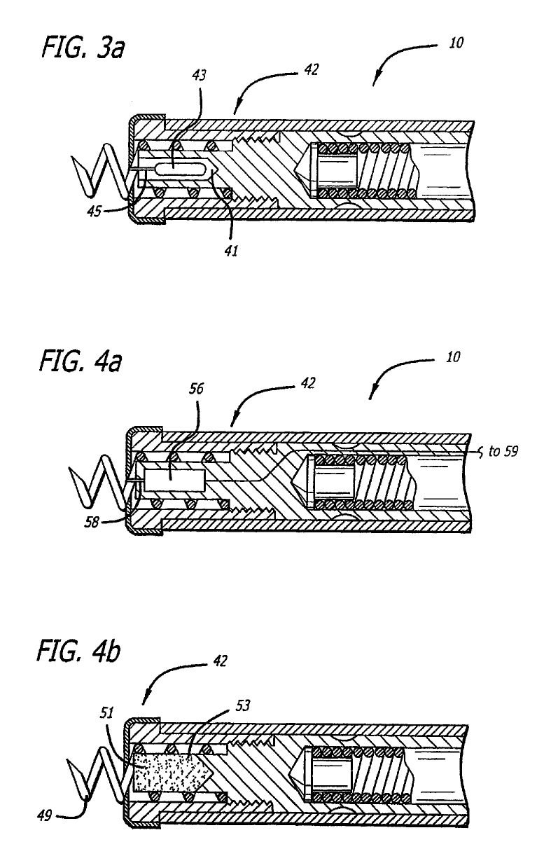

With reference to FIG. 3, in another embodiment, the lead 10 is an endocardial lead having the tip 42 including a distal chamber 44 which contains a matrix 46 loaded with the MMP inhibitor. The matrix is preferably a biocompatible silicone adhesive compound impregnated with the MMP inhibitor. In use, the lead 10 is inserted and the helix electrode 54 penetrates myocardial tissue. Upon implantation, bodily fluid in the vicinity of the selected myocardial location enters the chamber 44 through a screen 48, resulting in the elution of the MMP inhibitor from the matrix 46.

With reference to FIG. 3a, in another embodiment, the tip 42 of the lead 10 includes a distal chamber 41 which contains an osmotic pump 43, such as an ALZET osmotic pump (www.alzet.com). The pump 43 includes a reservoir (not shown) filled with a MMP inhibitor and an exit port 45 at the tip 42 of the lead 10. When the lead 10 is positioned in the body, the space in the chamber 41 surrounding the pump 43 is filled with fluid thereby activating operation of the pump 43. When activated, the fluid in the reservoir is released through the exit port 45.

With reference to FIG. 4, in another embodiment, the tip 42 of the endocardial lead 10 includes a drug eluting collar 50. The collar 50 may be a separate element secured to the end of the lead body 52 or may be integrally molded into the distal end of the lead body 52. The collar 50 may take any number of shapes or configurations that may be attached to or otherwise disposed on the distal end of the lead body 52. The collar 50 is typically in the shape of a ring that is attached over the exterior surface of the lead body 52 or a toroidal insert that is fitted within a cavity at the distal end lead body during manufacture. The collar 50 is generally positioned on the lead body 52 to allow the MMP inhibitor eluted from the collar 50 to come into contact with a target tissue proximate the electrode 54. Thus, the collar 50 is typically secured to the distal end of lead body 52.

To facilitate the elution of the MMP inhibitor, the collar 50 is constructed of a carrier material and the MMP inhibitor. The carrier material is typically a silicone rubber or a polymeric matrix, such as polyurethane. Generally, the carrier material is selected and formulated for an ability to incorporate the MMP inhibitor during manufacture and release the MMP inhibitor within the patient after implantation. The amount of the MMP inhibitor incorporated into collar 50 is determined by the effect desired, the potency of the MMP inhibitor, the rate at which the MMP inhibitor is released from the carrier material, as well as other factors that will be recognized by those skilled in the art.

The collar 50 in accordance with the present invention may be made by mixing (or dissolving, or melting). The MMP inhibitor will typically be mixed with uncured silicone rubber that includes, but is not necessarily limited to, two part liquid silicone rubbers, gum stock silicone rubbers, or medical adhesives used for creating or bonding silicone rubber components. The MMP inhibitor is added to the uncured silicone rubber in various quantities and following the mixing, the silicone rubber is cured and formed into the collar component for the delivery of the MMP inhibitor. Care should be taken that the method selected does not heat the mixture including the MMP inhibitor beyond a point that would destroy the MMP inhibitor. The collar 50 can be formed by any suitable process, including molding, extruding or other suitable processes recognized by those skilled in the art.

In another embodiment, the collar 50 of FIG. 4 is a microporous collar such as that described in U.S. Pat. No. 6,361,780, titled “Microporous Drug Delivery System,” assigned to Cardiac Pacemakers, Inc., the disclosure of which is hereby incorporated by reference.

In other embodiments, any exposed metallic or polymer component of the lead 10, such as the elongate body 32 (FIG. 2), the electrode 36 (FIG. 2) or the helix electrode 54 (FIG. 3) is coated with the MMP inhibitor. A typical method for coating these components includes applying a composition containing a polymer, a solvent, and the MMP inhibitor to the component using conventional techniques, for example, a dip-coating technique. Dip coating entails submerging all or part of the component or device into a polymer solution.

In another method, a plurality of pores, called “depots,” are formed in the outer surface of the component. The depots are sized and shaped to contain the composition to ensure that a measured dosage of the composition is delivered with the device to the specific treatment site. Depots formed on the components of the implantable device have a particular volume intended to be filled with the composition to increase the amount of the composition that can be delivered from the implantable device to the target treatment site.

The component can be made of a metallic material or an alloy such as, but not limited to, stainless steel, Nitinol, tantalum, nickel-titanium alloy, platinum-iridium alloy, gold, magnesium, or combinations thereof. The component may also be made from bioabsorbable or biostable polymers. A polymeric component should be chemically compatible with any substance to be loaded onto the component.

Depots, which may also be referred to as pores or cavities, can be formed in virtually any component structure at any preselected location. The location of depots within a component varies according to intended usage and application. Depots may be formed on the component by exposing the outer surface to an energy discharge from a laser, such as, but not limited to, an excimer laser. Alternative methods of forming such depots include but are not limited to, physical and chemical etching techniques. Such techniques are well known to one of ordinary skill in the art.

While the various embodiments of the lead configuration described thus far have been passive delivery devices, active delivery embodiments are contemplated. For example, with reference to FIG. 4a, in one active delivery embodiment, the tip 42 of the lead includes a pump reservoir 56 and delivery tube 58. The reservoir 56 is filled with the MMP inhibitor and delivery of the MMP inhibitor through the tube 58 is controlled by a pump controller 59 in the CRM housing. In an alternate configuration of this embodiment, the reservoir 56 may be located in the CRM housing and the delivery tube 58 extends the length of the lead from the tip to the CRM housing.

In another active delivery embodiment, electrophoresis is used to control delivery of the MMP inhibitor. With reference to FIG. 4b, in this embodiment a MMP inhibitor matrix 51 configured to release the MMP inhibitor when it is subjected to an electric field is carried in a chamber 53 in the tip 42 of the lead. The matrix 51 is surrounded by a helix tip electrode 49 configured to carry a non-pacing current. This current subjects the MMP inhibitor matrix 51 contained in the chamber 53 to an electric field causing release of the MMP inhibitor from the matrix.

### Stents

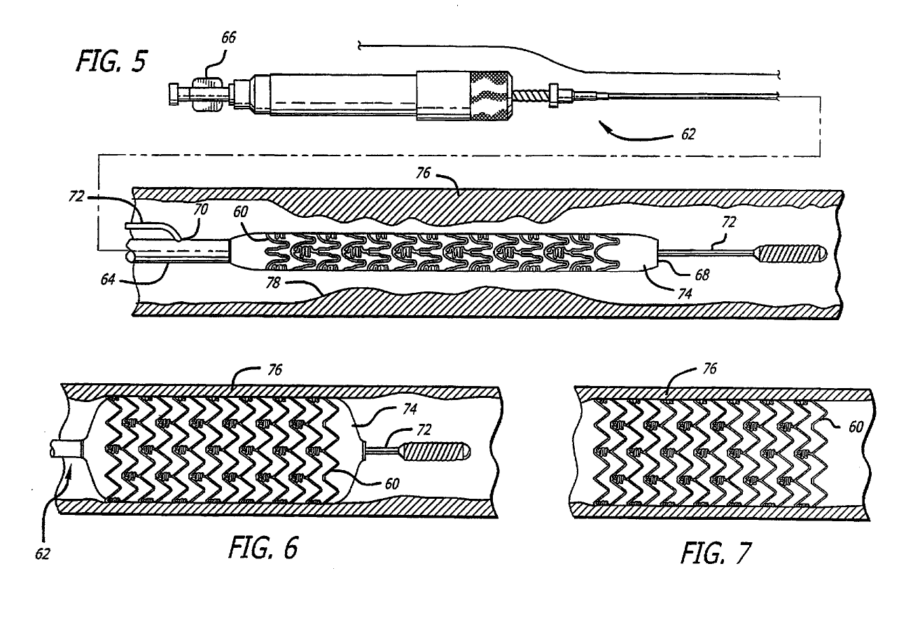

With reference to FIGS. 5, 6 and 7, in another embodiment of the invention, the implantable device is stent 60 positioned within the vascular system. As shown in FIG. 5, a stent 60 is mounted on a catheter assembly 62 which is used to deliver the stent to implant it in a body lumen, such as a coronary artery, peripheral artery, or other vessel or lumen within the body. The catheter assembly 62 includes a catheter shaft 64 which has a proximal end 66 and a distal end 68. The catheter assembly 62 is configured to advance through the patient's vascular system by advancing over a guide wire 72.

The catheter assembly 62 as illustrated in FIG. 5 is of a rapid exchange type which includes an RX port 70 where the guide wire 72 will exit the catheter. The distal end of the guide wire 72 exits the catheter distal end 68 so that the catheter advances along the guide wire on a section of the catheter between the RX port 70 and the catheter distal end 68. As is known in the art, the guide wire lumen which receives the guide wire 72 is sized for receiving various diameter guide wires to suit a particular application. The stent 60 is mounted on the expandable member 74 and is crimped tightly thereon so that the stent 60 and expandable member 74 present a low profile diameter for delivery through the arteries.

In FIG. 5, a partial cross-section of an artery 76 is shown with a small amount of plaque that has been previously treated by an angioplasty or other repair procedure. Stent 60 is used to repair a diseased or damaged arterial wall which may include plaque 78 as shown in FIG. 5, or a dissection, or a flap which are sometimes found in the coronary arteries, peripheral arteries and other vessels.

In an exemplary procedure to implant the stent 60, the guide wire 72 is advanced through the patient's vascular system by well known methods so that the distal end of the guide wire is advanced past the plaque or diseased area 78. Prior to implanting the stent, the cardiologist may wish to perform an angioplasty procedure or other procedure, i.e., atherectomy, in order to open the vessel and remodel the diseased area. Thereafter, the stent delivery catheter assembly 62 is advanced over the guide wire 72 so that the stent 60 is positioned in the target area. The expandable member or balloon 74 is inflated so that it expands radially outwardly and in turn expands the stent 60 radially outwardly until the stent 60 is apposed to the vessel wall. The expandable member 74 is then deflated and the catheter withdrawn from the patient's vascular system. The guide wire 72 is left in the lumen for post-dilatation procedures, if any, and subsequently is withdrawn from the patient's vascular system. As illustrated in FIG. 6, the balloon 74 is fully inflated with the stent 60 expanded and pressed against the vessel wall, and in FIG. 7, the implanted stent 60 remains in the vessel after the balloon has been deflated and the catheter assembly 62 (FIG. 6) and guide wire 72 have been withdrawn from the patient.

The stent 60 serves to hold open the artery after the catheter is withdrawn, as illustrated by FIG. 7. Due to the formation of the stent from an elongated tubular member, the undulating components of the stent are relatively flat in transverse cross-section. When the stent is expanded, it is pressed into the wall of the artery and accordingly does not interfere with the blood flow through the artery. The stent is pressed into the wall of the artery and will eventually be covered with endothelial cell growth which further minimizes blood flow interference.

In one embodiment, the entire surface of the stent 60 is coated to carry and deliver the MMP inhibitor. In another embodiment, portions of the surfaces of the stent 60, e.g., the tissue contacting portions, are coated to carry the MMP inhibitor. The stent 60 may be formed of either a metal or a polymer material and thus the methods available for medicating the stent 60 are the same as those described above with respect to the metallic and polymeric components of the lead configuration.

### Catheters

With reference to FIGS. 8-10, in another embodiment, transluminal catheters are configured to locally deliver a MMP inhibitor to cardiovascular regions that are made accessible by the catheters. In one embodiment, the catheters illustrated in FIGS. 8-10 are each a percutaneous transluminal vascular intervention (PTVI) device. Such a catheter enters a blood vessel of a patient through an incision site and advances through one or more blood vessels of the patient to reach a target intravascular or intracardiac region. In another embodiment, the catheters illustrated in FIGS. 8-10 are each an implantable lead, such as a lead for delivering electrical or other stimulation energy, delivering chemical or biological agents, and/or sensing physiologic signals.

As illustrated in FIG. 8, a catheter 80 has an elongate body 88 between a distal end portion 82 and a proximal end portion 84. Elongate body 88 is made of a polymer material such as medical grade silicone, polyurethane, Teflon, and polytetrafluoroethylene (PTFE). The distal end portion 82 is configured for intravascular or intracardiac placement and includes a distal tip 86. A lumen 90 extends within the elongate body 88 between the distal end portion 82 and the proximal end portion 84. The lumen 90 allows injection of a liquid agent including the MMP inhibitor to the intravascular or intracardiac region where the distal end portion 82 is placed.

As illustrated in FIG. 9, a catheter 94 has an elongate body 102 between a distal end portion 96 and a proximal end portion 98. Elongate body 102 is made of a polymer material such as medical grade silicone, polyurethane, Teflon, and PTFE. The distal end portion 96 is configured for intravascular or intracardiac placement and includes a distal tip 100. The distal end portion 96 includes a coating 104 that carries the MMP inhibitor and allows the MMP inhibitor to be released to the intravascular or intracardiac region where the distal end portion 96 is placed.

The coating 104 extends circumferentially completely (or at least partially) around the exterior surface of the catheter 94 at the distal end portion 96. When the distal end portion 96 is inserted into the body, the coating 104 dissolves and the MMP inhibitor is released. The time duration of the release of the MMP inhibitor is determined based on the length of time during which the distal end portion 96 is placed in the intravascular or intracardiac region during a catheterization procedure. The speed at which the coating 104 dissolves, and thus the MMP inhibitor is released, may be controlled based on the selection of the coating material and the concentration of the MMP inhibitor.

In one embodiment, the coating 104 includes substantially soluble particles dispersed in a substantially insoluble medium, such as biocompatible silicone rubber medical adhesive, other polymer, or other suitable biocompatible adhesive substance. The soluble particles are at least partially dissolvable when exposed to an aqueous substance such as blood or bodily fluids. The soluble particles include a MMP inhibitor and may also include a drug enhancer. When the coating 104 is exposed to an aqueous environment, the substantially soluble MMP inhibitor particles dissolve, providing sustained release of the MMP inhibitor into the surrounding tissue. The coating 104 is coated onto the distal end portion 96 during the manufacturing of the catheter 94. The coating 104 cures to adhere to the surface of the distal end portion 96 of catheter 94. Details relating to the coating formation are described in U.S. Pat. No. 6,584,363.

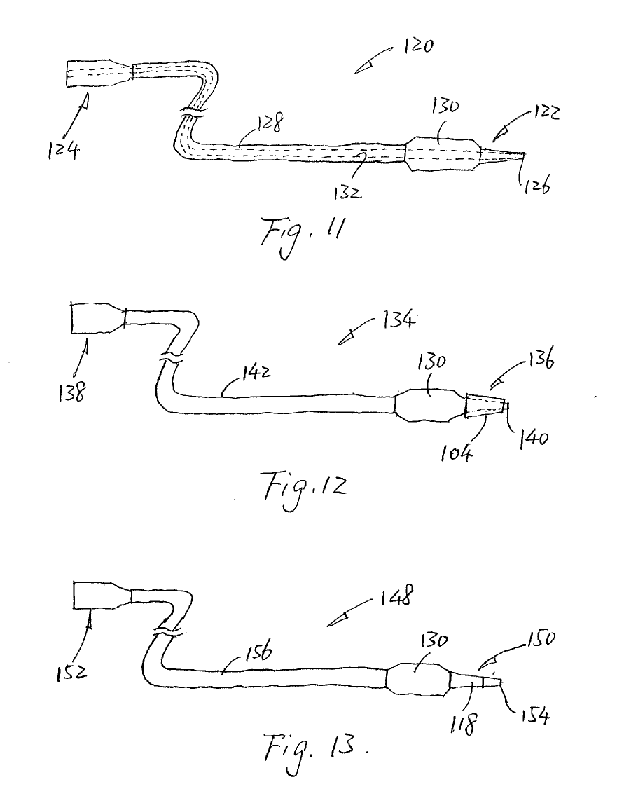

As illustrated in FIG. 10, a catheter 108 has an elongate body 116 between a distal end portion 110 and a proximal end portion 112. Elongate body 116 is made of a polymer material such as medical grade silicone, polyurethane, Teflon, and PTFE. Distal end portion 110 is configured for intravascular or intracardiac placement and includes a distal tip 114. The distal end portion 110 includes a drug eluting collar 118 that allows the MMP inhibitor to elute to the intravascular or intracardiac region where the distal end portion 110 is placed.

To facilitate the elution of the MMP inhibitor, the collar 118 is constructed of a carrier material and the MMP inhibitor. Examples of the carrier material include a silicone rubber or a polymeric matrix, such as polyurethane. Generally, the carrier material is selected and formulated for an ability to incorporate the MMP inhibitor during manufacture and release the MMP inhibitor when the distal end portion 110 is within the patient. The amount of the MMP inhibitor incorporated into collar 118 is determined by the effect desired, the potency of the MMP inhibitor, the rate at which the MMP inhibitor is released from the carrier material, as well as other factors that will be recognized by those skilled in the art.

In various embodiments, the collar 118 is made by mixing (or dissolving, or melting). The MMP inhibitor is mixed with uncured silicone rubber. In one embodiment, two part liquid silicone rubbers, gum stock silicone rubbers, or medical adhesives are used for creating or bonding silicone rubber components. The MMP inhibitor is added to the uncured silicone rubber in various quantities and following the mixing, the silicone rubber is cured and formed into the collar component for the delivery of the MMP inhibitor. Care should be taken that the method selected does not heat the mixture including the MMP inhibitor beyond a point that would destroy the MMP inhibitor. The collar 118 can be formed by any suitable process, including molding, extruding or other suitable processes recognized by those skilled in the art. In another embodiment, the collar 118 is a microporous collar, such as described in U.S. Pat. No. 6,361,780.

With reference to FIGS. 11-13, in another embodiment, catheters used for angioplasty are configured to locally deliver the MMP inhibitor to an intravascular region where the angioplasty is performed. When the angioplasty is performed in a coronary artery, the catheters are also referred to as percutaneous transluminal coronary angioplasty (PTCA) devices.

As illustrated in FIG. 11, a catheter 120 has an elongate body 128 between a distal end portion 122 and a proximal end portion 124. Elongate body 128 is made of a polymer material such as medical grade silicone, polyurethane, Teflon, and PTFE. Distal end portion 122 is configured for intravascular placement and includes a distal tip 126. The distal end portion 122 includes an angioplasty device 130 proximal to the distal tip 126. In various embodiments, angioplasty device 130 allows for application of an angioplastic therapy such as vascular dilatation, stent delivery, brachytherapy (radiotherapy), atherectomy, or embolic protection. In one embodiment, the angioplasty device 130 includes an adjustable portion that has controllable expandability and contractibility. In one specific embodiment, angioplasty device 130 includes a balloon that is inflated and deflated through a passageway longitudinally extending within elongate body 128 and connected between the chamber of the balloon and a connector at proximal end portion 124. The balloon is inflatable using an air pump connected to that connector. In one embodiment, the distal tip 126 is a tapered tip that facilitates the insertion of the catheter 120 into a blood vessel. Proximal end portion 124 includes a structure that accommodates all the mechanical connection and access requirements, which depend on the function of the angioplasty device 130.

A lumen 132 extends within the elongate body 128 between the distal end portion 122 and the proximal end portion 124. The lumen 132 allows injection of the MMP inhibitor to the vascular location where the distal end portion 122 is placed.

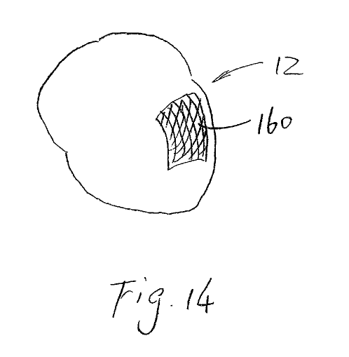

As illustrated in FIG. 12, a catheter 134 has an elongate body 142 between a distal end portion 136 and a proximal end portion 138. Elongate body 142 is made of a polymer material such as medical grade silicone, polyurethane, Teflon, and PTFE. Distal end portion 136 is configured for intravascular placement and includes a distal tip 140. The distal end portion 136 includes the angioplasty device 130 proximal to the distal tip 140. In one embodiment, the distal tip 140 is a tapered tip that facilitates the insertion of the catheter 134 into a blood vessel. Proximal end portion 138 includes a structure that accommodates all the mechanical connection and access requirements, which depend on the function of the angioplasty device 130. The distal end portion 136 includes the coating 104 that carries the MMP inhibitor and allows the MMP inhibitor to be released to the vascular location where the distal end portion 136 is placed.

As illustrated in FIG. 13, a catheter 148 has an elongate body 156 between a distal end portion 150 and a proximal end portion 152. Elongate body 156 is made of a polymer material such as medical grade silicone, polyurethane, Teflon, and PTFE. Distal end portion 150 is configured for intravascular placement and includes a distal tip 154. The distal end portion 154 includes the angioplasty device 130 proximal to the distal tip 154. In one embodiment, the distal tip 154 is a tapered tip that facilitates the insertion of the catheter 148 into a blood vessel. Proximal end portion 152 includes a structure that accommodates all the mechanical connection and access requirements, which depend on the function of the angioplasty device 130. The distal end portion 150 includes the drug eluting collar 118 that allows the MMP inhibitor to elute to the vascular location where the distal end portion 150 is placed.

For illustrative but not restrictive purposes, catheters shown in FIGS. 8-13 are each configured for localized delivery of the MMP inhibitor to the region where the distal end portion of the catheter is placed. In general, one or more portions of a catheter each include a coating carrying the MMP inhibitor, such as coating 104, and/or a drug eluting collar carrying the MMP inhibitor, such as collar 118. In one embodiment, one or more surface portions of the catheter each include the “depots”. In various embodiments, the coating 104 and/or the collar 118 are incorporated into one or more portions of a catheter using a procedure that is substantially identical or similar to the procedure of incorporating a coating or a collar into a lead, as discussed above.

### Heart Patches

FIG. 14 is an illustration of an embodiment of a heart patch 160 carrying a MMP inhibitor. In the illustrated embodiment, heart patch 160 is an epicardial patch having one or more MMP inhibitors coated on and/or embedded in the matrix of the patch or at least a portion thereof. In various embodiments, heart patch 160 is attached to heart 12 to provide cardiac support or ventricular remodeling control, such as to resist myocardial dilation in heart 12.

In one embodiment, the entire surface of heart patch 160 is coated to carry and deliver the MMP inhibitor. In another embodiment, portions of the surfaces of heart patch 160, e.g., the portions configured to contact the epicardial surface, are coated to carry the MMP inhibitor. Heart patch 160 may be formed of either a metal or a polymer material and thus the methods available for medicating the heart patch are the same as those described above with respect to the metallic and polymeric components of the lead configuration. In one embodiment, heart patch 160 is made of a biodegradable material that is absorbed by the body after providing support to heart 12 for a certain period of time and the MMP inhibitor has been eluted.

### Compositions, Dosages and Routes of Administration

The MMP inhibitors of the invention may be employed in conjunction with other therapies, e.g., therapies for ischemia or arrhythmias. The amount of MMP inhibitor and/or other drugs which are exogenously administered will vary depending on various factors

Administration of the agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses.

The formulations may, where appropriate, be conveniently presented in discrete unit dosage forms and may be prepared by any of the methods well known to pharmacy. Such methods may include the step of bringing into association the agent with liquid carriers, solid matrices, semi-solid carriers, finely divided solid carriers or combinations thereof, and then, if necessary, introducing or shaping the product into the desired delivery system.

Pharmaceutical formulations containing the agents of the invention can be prepared by procedures known in the art using well known and readily available ingredients. For example, the agent can be formulated with common excipients, diluents, or carriers. Examples of excipients, diluents, and carriers that are suitable for such formulations include the following fillers and extenders such as starch, sugars, mannitol, and silicic derivatives; binding agents such as carboxymethyl cellulose, HPMC and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols. The formulations can include buffering agents such as calcium carbonate, magnesium oxide and magnesium carbonate, as well as, inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and gelatin, microcrystalline cellulose, or sodium lauryl sulfate, or liquid vehicles such as polyethylene glycols (PEGs) and vegetable oil.

The pharmaceutical formulations of the agents of the invention can also take the form of an aqueous or anhydrous solution or dispersion, or alternatively the form of an emulsion or suspension.

The compositions according to the invention can also contain thickening agents such as cellulose and/or cellulose derivatives. They can also contain gums such as xanthan, guar or carbo gum or gum arabic, or alternatively polyethylene glycols, bentones and montmorillonites, and the like.

It is possible to add, if necessary, an adjuvant chosen from antioxidants, surfactants, other preservatives, film-forming, keratolytic or comedolytic agents, perfumes and colorings. Also, other active ingredients may be added, whether for the conditions described or some other condition.

Additionally, the agents are well suited to formulation as sustained release dosage forms and the like. The coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., a stent, epicardial patch, lead, and the like.

The formulations and compositions described herein may also contain other ingredients such as antimicrobial agents, or preservatives. Furthermore, as described herein the active ingredients may also be used in combination with other therapeutic agents or therapies.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A system for locally delivering one or more agents to a treatment site in or near a heart, the system comprising:
an implantable device including at least a portion configured to be positioned in the treatment site and to carry and locally deliver one or more inhibitors of one or more matrix metalloproteinases (MMPs) to the treatment site, the one or more MMP inhibitors inhibiting at least one of MMP-1, MMP-2, MMP-8, MMP-13 or MT-1.

2. The system of claim 1 wherein the implantable device comprises a lead including a proximal end portion, a distal end portion, an elongate body coupled between the proximal end portion and the distal end portion, and a lumen extending in the elongate body from the proximal end portion to the distal end portion, the lumen configured to deliver the one or more MMP inhibitors.

3. The system of claim 1 wherein the implantable device comprises a lead including a proximal end portion, a distal end portion, an elongate body coupled between the proximal end portion and the distal end portion, and a lumen extending in the elongate body from the proximal end portion to the distal end portion, the distal end portion including a coating adapted to carry and deliver the one or more MMP inhibitors.

4. The system of claim 1 wherein the implantable device comprises a lead including a proximal end portion, a distal end portion, an elongate body coupled between the proximal end portion and the distal end portion, and a lumen extending in the elongate body from the proximal end portion to the distal end portion, the distal end portion including a drug eluting collar adapted to carry and deliver the one or more MMP inhibitors.

5. The system of claim 1 wherein the implantable device comprises a stent including at least one surface portion coated with a material including the one or more MMP inhibitors.

6. The system of claim 1 wherein the implantable device comprises a heart patch including at least one surface portion coated with a material including the one or more MMP inhibitors.

7. The system of claim 1 wherein the one or more inhibitors are selective inhibitors of at least one of MMP-1, MMP-2, MMP-8, MMP-13 or MT-1.

8. The system of claim 7 wherein the one or more inhibitors are selective inhibitors of at least two of MMP-1, MMP-2, MMP-8, MMP-13 or MT-1.

9. The system of claim 1 wherein the device delivers two or more inhibitors of at least two of MMP-1, MMP-2, MMP-8, MMP-13 or MT-1.

10. The system of claim 1 wherein at least one inhibitor is not a tissue inhibitor of a matrix metalloproteinase (TIMP).

11. A system for locally delivering one or more agents to a treatment site in or near a heart, the system comprising:
a percutaneous transluminal catheter including at least a portion configured to be positioned in the treatment site and to carry and locally deliver one or more inhibitors of one or more MMPs to the treatment site, the one or more MMP inhibitors inhibiting at least one of MMP-1, MMP-2, MMP-8, MMP-13 or MT-1.

12. The system of claim 11 wherein the percutaneous transluminal catheter comprises a proximal end portion, a distal end portion, an elongate body coupled between the proximal end portion and the distal end portion, and a lumen extending in the elongate body from the proximal end portion to the distal end portion, the lumen configured to deliver the one or more MMP inhibitors.

13. The system of claim 11 wherein the percutaneous transluminal catheter comprises a proximal end portion, a distal end portion, and an elongate body coupled between the proximal end portion and the distal end portion, the distal end portion including a coating adapted to carry and deliver the one or more MMP inhibitors.

14. The system of claim 11 wherein the percutaneous transluminal catheter comprises a proximal end portion, a distal end portion, and an elongate body coupled between the proximal end portion and the distal end portion, the distal end portion including a drug eluting collar adapted to carry and deliver the one or more MMP inhibitors.

15. The system of claim 11 wherein the percutaneous transluminal catheter comprises a proximal end portion, a distal end portion, and an elongate body coupled between the proximal end portion and the distal end portion, the distal end portion including an angioplasty device.

16. The system of claim 15 wherein the angioplasty device is adapted to perform at least one of vascular dilatation, stent delivery, brachytherapy, atherectomy, and embolic protection.

17. The system of claim 11 wherein the one or more inhibitors are selective inhibitors of at least one of MMP-1, MMP-2, MMP-8, MMP-13 or MT-1.

18. The system of claim 17 wherein the one or more inhibitors are selective inhibitors of at least two MMP-1, MMP-2, MMP-8, MMP-13 or MT-1.

19. The system of claim 11 wherein the device delivers two or more inhibitors of at two of MMP-1, MMP-2, MMP-8, MMP-13 or MT-1.

20. The system of claim 11 wherein at least one inhibitor is not a tissue inhibitor of a matrix metalloproteinase (TIMP).

21. A method for treating a myocardial region including at least a portion of an injured area, comprising:
delivering pacing pulses to the myocardial region of a mammal through one or more electrodes of a plurality of pacing electrodes on a lead; and
delivering one or more inhibitors of one or more MMPs through a lumen in the lead in an amount effective to prevent or inhibit remodeling.

22. A method for treating a myocardial region including at least a portion of an injured area, comprising:
delivering pacing pulses to the myocardial region of a mammal through one or more electrodes of a plurality of pacing electrodes on a lead; and
delivering one or more inhibitors of one or more MMPs through a lumen in the lead in an amount effective to enhance pacing.

23. The method of claim 21 or 22 wherein the one or more inhibitors inhibit at least one of MMP-1, MMP-2, MMP-8, MMP-13 or MT-1.

24. The method of claim 21 or 22 wherein the one or more inhibitors are selective inhibitors of at least two MMP-1, MMP-2, MMP-8, MMP-13 or MT-1.

25. The method of claim 20 or 21 wherein the device delivers two or more inhibitors of at least two of MMP-1, MMP-2, MMP-8, MMP-13 or MT-1.

26. The method of claim 21 or 22 wherein the one or more inhibitors are selective inhibitors of at least one of MMP-2, MMP-8, MMP-13 or MT-1.

27. The method of claim 21 or 22 wherein at least one inhibitor is not a TIMP.

28. A method for treating a myocardial region including at least a portion of an injured area, comprising:
introducing to a mammal via a catheter a stent which stent is coated with an effective amount of one or more inhibitors of one or more MMPs; and
delivering an effective amount of one or more inhibitors of one or more MMPs via a lumen in the catheter, wherein the inhibitors in the stent may be the same or different than the inhibitors delivered by the catheter.

29. A method for treating a myocardial region including at least a portion of an injured area, comprising:
introducing to a mammal a heart patch coated with an effective amount of one or more inhibitors of one or more MMPs.

30. A system for a heart having a myocardial infarct region, comprising:
an implantable agent delivery device adapted to carry and release one or more inhibitors of one or more MMPs to a cardiac region including at least a portion of a myocardial infarct region; and
an implantable cardiac rhythm management (CRM) device coupled to the implantable agent delivery device, the implantable CRM device including:
a pacing circuit to deliver pacing pulses to the cardiac region; and
a pacing controller adapted to control the delivery of the pacing pulses, wherein the agent delivery device comprises a pacing lead connected to the implantable CRM device, the pacing lead including at least one electrode to be placed in or near the myocardial infarct region.

31. The system of claim 24 wherein the pacing lead comprises the implantable agent delivery device.

32. The system of claim 31 wherein the pacing lead comprises a proximal end portion connected to the implantable CRM device and a distal end portion configured to be placed in or near the myocardial infarct region, the distal end portion including a coating adapted to carry and deliver the one or more inhibitors.

33. The system of claim 31 wherein the pacing lead comprises a proximal end portion connected to the implantable CRM device and a distal end portion configured to be placed in or near the myocardial infarct region, the distal end portion including a drug eluting collar adapted to carry and deliver the one or more inhibitors.

34. A system to treat a myocardial infarct, comprising:
an implantable agent delivery device adapted to contain one or more inhibitors of one or more MMPs and to release the one or more inhibitors to a cardiac region including at least a portion of a myocardial infarct region, wherein the device includes a stent; and
a catheter having a lumen adapted for delivery of one or more inhibitors of one or more MMPs.
